(19) United States
(12) Patent Application Publication
(10) Pub. No.: US 2002/0150882 A1
(43) Pub. Date: Oct. 17, 2002

(54) Antibody specific to CD14 and uses thereof

(76) Inventors: Andrew Devitt, Staffordshire (GB); Christopher Darrell Gregory, Nottingham (GB); Jaspal Sanghera, Cambridge (GB); Sarah Elizabeth Pierce, Nottingham (GB)

Correspondence Address: Mathews, Collins, Shepherd & Gould, P.A., Suite 306, 100 Thanet Circle, Princeton, NJ, 08540, US

(21) Appl. No.: 09/835,756

(22) Filed: Apr. 16, 2001

(51) Int. Cl. C12Q 1/70; G01N 33/567; A61K 39/395; C07K 16/18

(52) U.S. Cl. 435/5; 435/7.21; 530/388.1; 424/145.1

(57) ABSTRACT

A method of detecting CD14 expressing cells using at least one of Ce3, R7 or R29. Ce3, R7 or R29 being capable of being expressed from bacterial strains having the accession numbers MNIMB 41088, NCIMB 41086 and NCIMB 41087 respectively.

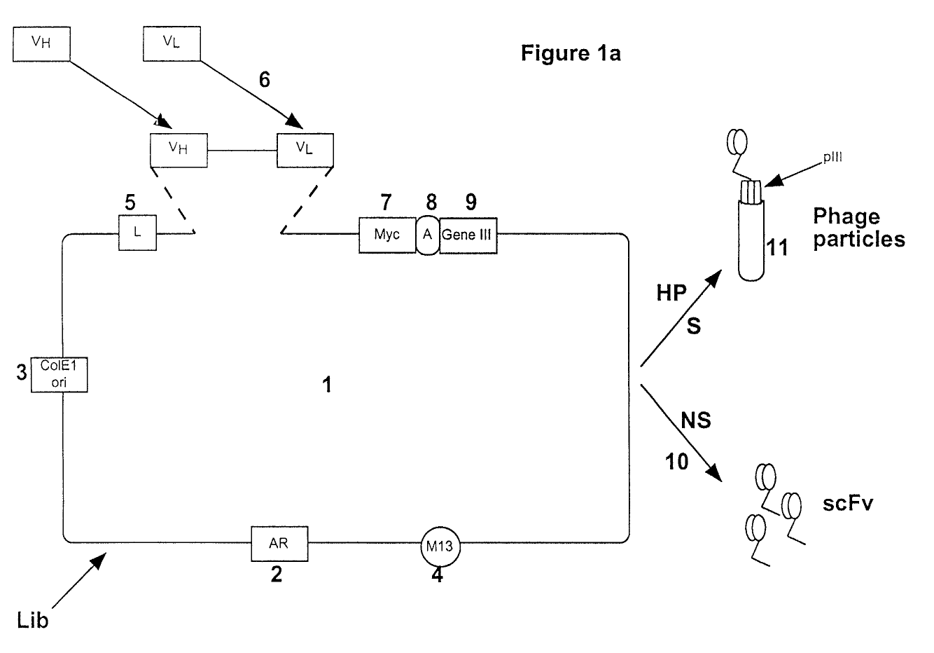

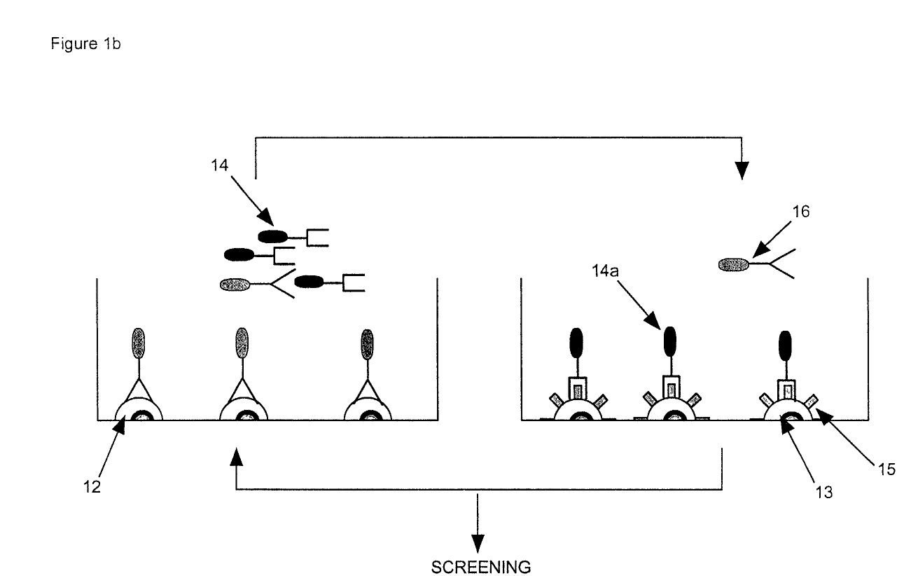

To be accompanied, when published by FIG. 1a of the drawings.

This invention relates to an antibody specific to CD14 and uses thereof.

CD14 is a glycosylphosphatidylinositol-linked plasma membrane glycoprotein. It is known that this receptor is involved in the binding of bacterial lipopolysaccharides (LPS) and other microbial products, and therefore triggering inflammatory responses to infection. Indeed, overstimulation of CD14, by LPS, can cause septic shock syndrome.

CD14 is expressed largely on monocytes, macrophages find to a lesser extent on granulocytes. Since the original discovery of the function of CD14 as a mediator of inflammation it has been discovered to have a further action in mediating the clearance of apoptotic cells (AC) by macrophages (Devitt et al., 1998). This is surprising as AC removal is a non-inflammatory process.

It is desirable to identify a reagent that can identify and/or manipulate the actions of CD14. The reagent may have uses in the treatment of inflammatory disease and in the indirect monitoring of apoptosis.

Accordingly, the invention relates to an antibody reactive with CD14 and uses thereof. According to a first aspect the invention comprises a method of detecting CD14 comprising using at least one of: Ce3, R7 or R29 to detect CD14.

It is believed that CD14 plays a role in AC clearance by phagocytes and can modulate the AC clearance process and is also implicated in inflammatory responses.

According to another aspect the invention comprises a method of detecting CD14 comprising recognising CD14 using at least one antibody capable of being expressed from the cDNA isolated from the bacterial strains having accession number NCIMB 41086, NCIMB 41087, or NCIMB 41088.

The antibody, Ce3, R7 or R29, to CD14, may preferably be a monoclonal antibody (mAb).

According to another aspect the invention comprises a method of matching an antibody which detects CD14 comprising using an antibody library.

Preferably, the antibody is produced using, or derived from a phage antibody library. The antibody may comprise a whole phage. The antibody may also be produced using, derived from and comprise a single chain Fv (variable fragment) molecule. Conventional Ab technology may also be usefully employed.

Single chains have less material and are likely to have interactions that are potentially more specific than are whole phage antibodies.

The generation/selection of useful phage antibodies against CD14 (Ø-Abs) from a Ø-Ab library is preferably achieved without using all immunisation step as would be necessary with conventional Ab techniques. This makes them more widely applicable than if an immunological reaction was necessary to produce them.

Preferably the antibodies selected are at least one from the group Ce3, R7 and R29. The invention can be, in another aspect, considered to be a method for identifying antibodies which are specific to CD14 comprising raising such antibodies from a phage library and screening them for activity against CD14.

The antibodies may be produced in a ‘suppressor strain’ of E. coli with helper phage e.g. M13. This produces the phage antibody library. Once a suitable Ab has been identified, and possibly screened to ensure that it does really work in binding to structures involved in clearance (and/or identify ACs specifically), we can select out from the phage library (e.g. by extracting relevant DNA) to produce a single chain, for example using a non-suppressor strain of E. coli.

We have identified 3 antibodies specific for the lipopolysaccharide receptor, CD14, using phage display technology; Ce3, R7, Fund R29.

The antibodies may influence other receptors, possibly CD11c/CD18 integrin (CR4), or another lipopolysaccharide receptor, or the Toll-like receptors (TLRs).

Ce3 is an antibody obtainable by the method of the invention and by the cDNA deposited at National Centre for Industrial Food and Marine Bacteria, 23 St. Machar Drive, Aberdeen, AB24 3RY (Accession No. NCIMB 41088) on Mar. 16, 2001. Ce3 is capable of binding cell-associated CD14 e.g. surface expressed CD14 that is not already bound to lipopolysaccharide or a ligand on ACs.

R7 and R29 are antibodies obtainable by the method of the invention and by the cDNA deposited at National Centre for Industrial, Food and Marine Bacteria, 23 St. Machar Drive, Aberdeen, AB24 3RY (Accession Nos. NCIMB 41086 and NCIMB 41087 respectively) on Mar. 16, 2001 on R7 and 29 are capable of binding non-cell-associated CD14 only (i.e. soluble CD14).

It is believed that the antigen binding site of Ce3, R7 or R29 is key to generating non-antibody substances equivalent to, or complementary to the binding site of Ce3, R7 or R29 could have a use as a diagnostic, therapeutic, or imaging reagents or other reagents.

By “substances equivalent to” we mean a substance that is reactive with CD14 and has substantially the same amino acid sequence.

We may also generate non-antibody substances using the sense or antisense strand of the DNA comprising the genetic information of Ce3, R7 or R29 to generate peptide substances for use as diagnostic, therapeutic or imaging reagents or other reagents.

When we say Ce3 we mean any antibody that is like Ce3, for example any antibody that can bind to CD14 in substantially the same manner as Ce3 with substantially the same amino acid sequence and/or same epitope on CD14 or cDNA sequence, and/or to substantially the same epitope on CD14.

When we say Ce3 we also mean any molecule with 10-100% sequence identity and/or homology with Ce3 for example 20-100% or 30-100% or 40-100% or 50-100% or 60-100% or 70-100% 80-100% or 90-100% sequence identity and/or homology with Ce3.

When we say R7 and R29 we mean any antibody that is like them, for example any antibody that can bind to soluble CD14 in substantially the same manner and/or having substantially the same amino acid sequence and/or cDNA sequence, and/or to substantially the same epitope on CD14.

When we say R7 we also mean any molecule with 10-100% sequence identity and/or homology with, for example, 20-100% or 30-100% or 40-100% or 50-100% or 60-100% or 70-100% or 80-100% or 90-100% sequence identity or homology with R7.

When we say R29 we also mean any molecule with 10-100% sequence identity and/or homology with R29, for example, 20-100% or 30-100% or 40-100% or 50-100% or 60-100% or 70-100% or 80-100% or 90-100% sequence identity and/or homology with R29.

In addition to being able to bind to CD14, the antibody Ce3 (or R7 or R29) may influence the clearance of apoptotic cells by phagocytes. These two abilities may be linked.

We may arrange for these antibodies obtained from the phage library to be species specific. We may re-engineer the Ø-Ab back to a conventional antibody, possibly a human antibody of the IgG form. We may conjugate it with a useful other molecule.

According to another aspect the invention comprises the use of Ce3 in the identification of non-inflammatory drugs, Ce3 being able to bind to a receptor of apoptotic cells, CD14 (on phagocytes), which engage with ACs to cause apoptotic-cell clearance, or able otherwise to influence AC-phagocyte clearance events or other CD14 functions (e.g. proinflammation).

According to another aspect the invention comprises the use of Ce3 in the preparation of medicament for the treatment of an inflammatory disease or condition, Ce3 being able to bind to a receptor of apoptotic cells, CD14 (on phagocytes), which engage ACs to cause apoptotic-cell clearance, or which are able to influence AC-phagocyte clearance events or other CD14 functions (e.g. proinflammation).

The Ce3 may be in a pure or isolated form. The pure isolated Ce3 may be administered at a concentration of between 0-10000 mg/ml.

According to another aspect the invention comprises a method of diagnosis of the presence of CD14-expressing cells comprising applying Ce3 to cells of interest of a test area and observing a signal from the test area, Ce3 being able to bind to CD14 on phagocytes, CD14 being involved in apoptotic-cell recognition by phagocytes in the clearance process of apoptotic cells and in the binding of bacterial LPS and other microbial products; and having a marker adapted to provide said signal.

The diagnosis may be performed in vitro, or in vivo. The diagnosis may be performed on histological tissue samples.

When the method of diagnosis is performed in vivo it may comprise imaging a portion of the patient's body (human or animal).

According to another aspect the invention comprises Ce3 or R7 or R29 as a diagnostic reagent for use in a method of diagnosing the presence of CD14 expressing cells, Ce3 being able to bind to CD14 on phagocytes and R7 and R29 being able to bind soluble CD14; and which have a marker on them, or a site to bind a marker, to provide a signal to indicate the presence of Ce3 or R7 or R29.

The marker may be a fluorochrome, or radio-label, or MRI label, or any detectable marker, or a site adapted to bind one of them.

The diagnostic reagent may be an imaging reagent.

According to another aspect the invention comprises Ce3, R7 or R29 as a probe for use in a method of detecting the presence of CD14 expressing cells, and R7 and R29 being able to bind soluble CD14; and which have a marker on them, or a site to bind a marker, to provide a signal to indicate the presence of Ce3 or R7 or R29.

The marker may be a fluorochrome, or radio label, or MRI label, or any detectable marker, or a site adapted to bind one of them.

The probe may be an imaging reagent.

According to another aspect, the invention comprises Ce3, R7 or R29 as a medicament in the treatment of any disease or disorder in which monitoring the levels of CD14-expressing cells may be useful.

According to another aspect the invention comprises Ce3, R7 or R29 as a medicament in the treatment of any disease or disorder in which suppression of inflammation may be an effective therapy.

According to another aspect the invention comprises Ce3, R7 or R29 as a medicament in the treatment of any disease or disorder in which the modulation of function of CD14-expressing cells may be an effective therapy.

According to another aspect the invention comprises Ce3, R7 or R29 as a therapeutic for use in any disease or disorder where monitoring the levels of CD14-expressing cells, or suppression of inflammation, or the modulation of function of CD14-expressing cells may be an effective therapy.

According to another aspect the invention comprises a method of treating a disease or disorder by administering a medicament which contains Ce3 and a pharmaceutically acceptable carrier.

According to another aspect the invention comprises the use of Ce3 in the preparation of a medicament for the treatment of a disease or disorder.

Ce3 is able to bind to CD14, including CD14 on phagocytes.

The disease or disorder may be from (but not limited to) the list:

Cancer;

Autoimmune disease;

Neurodegenerative diseases;

Stroke;

Infertility;

Myocardial infarction;

Inflammatory disorders;

Any condition where suppression of inflammation may be an effective therapy;

Any condition where modulation of function of CD14-expressing cells may be an effective therapy.

According to another aspect the invention comprises a method of prognosis of a treatment for a disease or disorder, the method comprising administering a treatment for the disease or disorder; waiting a time sufficient for the treatment to have a measurable effect; administering an imageable or otherwise detectable reagent, and in which the imageable or otherwise detectable reagent comprises Ce3, and wherein the reagent has a detectable signal or has a binding site for a signal-generating marker that is also administered, imaging or otherwise analysing the signal from the site of the patient that has the disorder or disease to evaluate the level of CD14 expressing cells at the site, thereby enabling a prognosis for the patient to be evaluated by evaluating the efficacy of the treatment.

Comparison of the results ‘before and after’ treatment may be necessary to confirm any effects of the treatment.

Instead of practising the prognosis indication on the body of a patient it could be practised in vitro—for example on a tissue sample or a blood or other fluid or biological sample.

According to another aspect the invention comprises a prognosis indicating or imaging reagent having Ce3 and a signal marker, or a binding site for a signal marker.

Ce3 is, of course, capable of binding to CD14 on phagocytes. CD14 is able to engage ACs to cause apoptotic cell clearance, and is implicated in inflammatory responses.

The signal marker may be in the same reagent as Ce3 or other reagents (e.g. be part of them), or it may comprise a separate reagent, the invention may be a kit having both signal-producing reagent and prognosis/imaging antibody (Ce3). Of course the prognosis indication could occur in vivo or in vitro.

The invention will now be described by way of example with reference to the accompanying drawings, of which:

FIGS. 1a, 1b and 1e schematically illustrate a phage antibody library construct and a method for isolating and identifying phage antibodies in accordance with the invention;

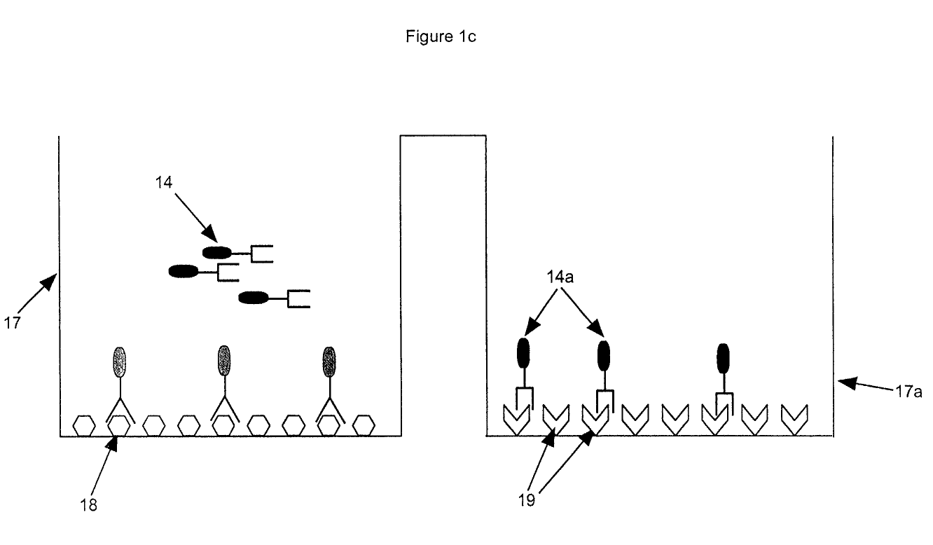

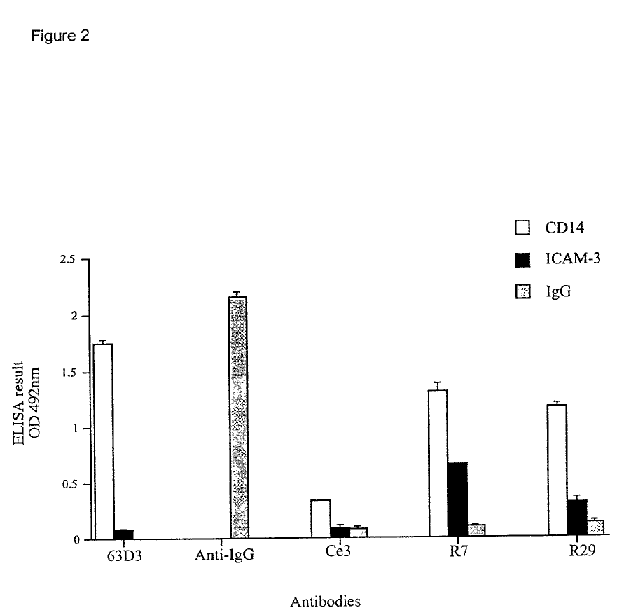

FIG. 2 shows the results of an ELISA where CD14-Fc, ICAM3-Fc and IgG were coated on a plate and screened phage antibodies are allowed to bind to CD14-Fc, ICAM3-Fc or IgG;

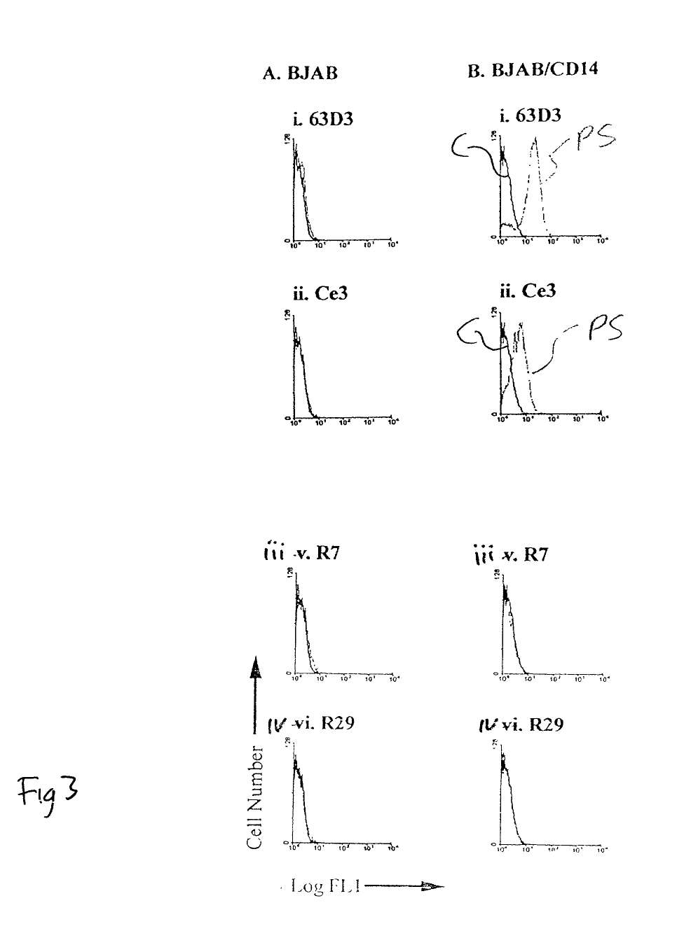

FIG. 3 shows FLOW CYTOMETRIC profile results of a range of Abs binding on two cell types: BJAB and BJAB-CD14 (where CD14 is exogenously expressed on the cell surface);

FIG. 4 shows FLOW CYTOMETRIC profile results of a range of Abs binding on two cell types K562 (human erythroleukaemia cells) and CHO (Chinese Hamster Ovary cells) exogenously expressing human CD14;

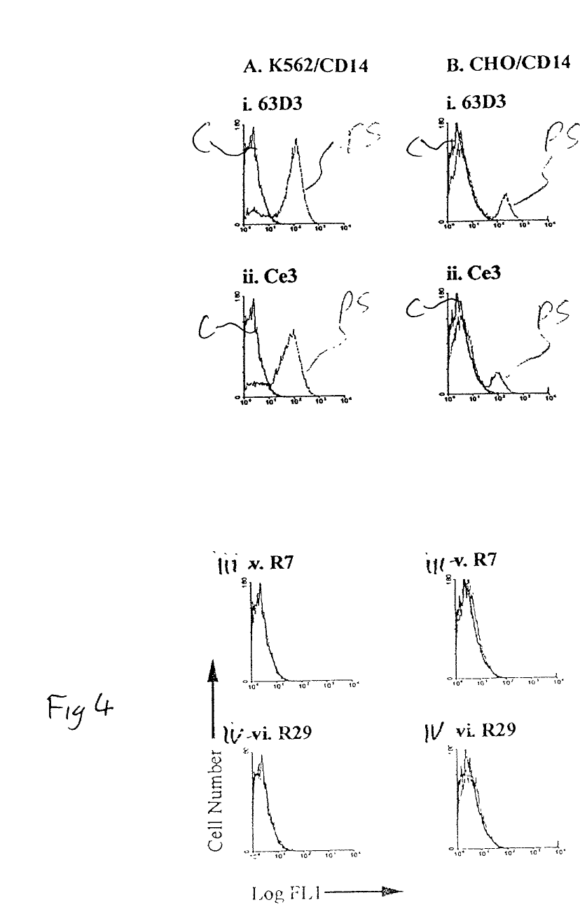

FIG. 5 shows FLOW CYTOMETRIC profile results of a range of antibodies binding to THP-1 cells stimulated to express CD14 with dihydroxy vitamin D3;

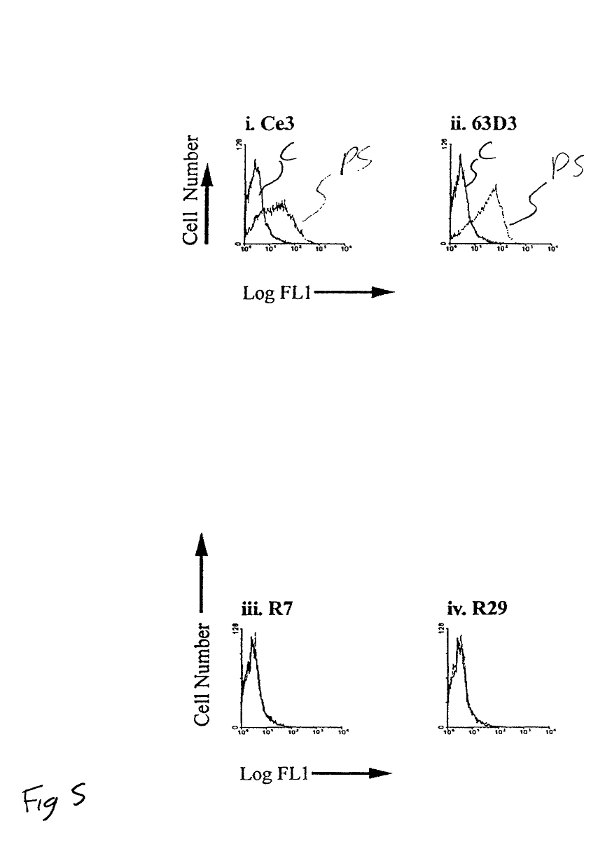

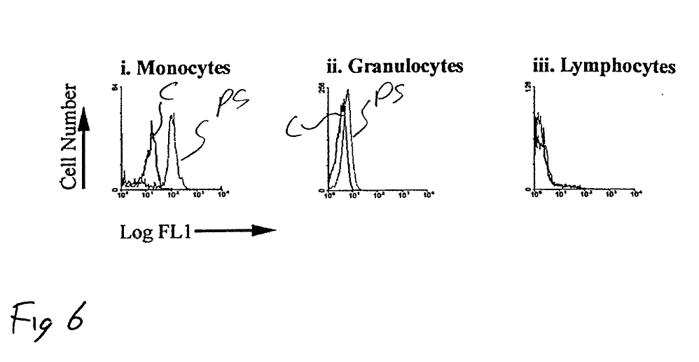

FIG. 6 shows FLOW CYTOMETRIC profile results of Ce3 (as an scFv) binding to monocytes granulocytes and lymphocytes.

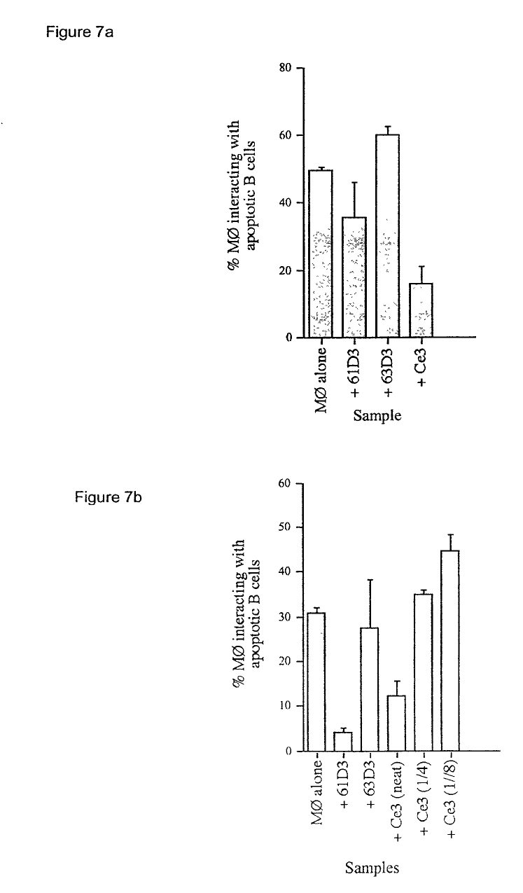

FIG. 7 shows the ability of Ce3 to inhibit the interaction of ACs with macrophages and therefore block the clearance of ACs.

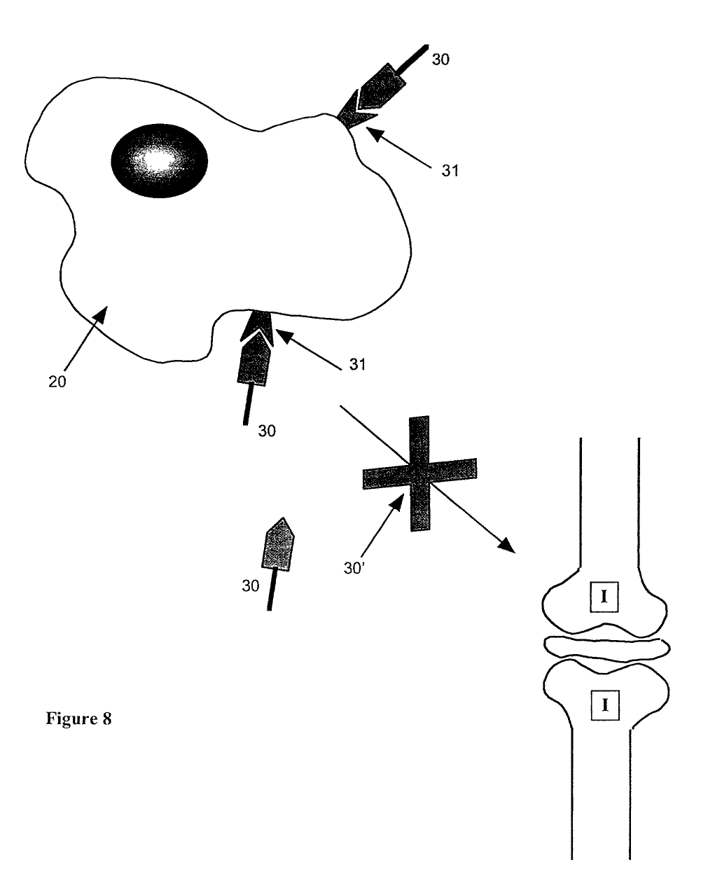

FIG. 8 schematically illustrates the use of Ce3 as a ligand mimetic;

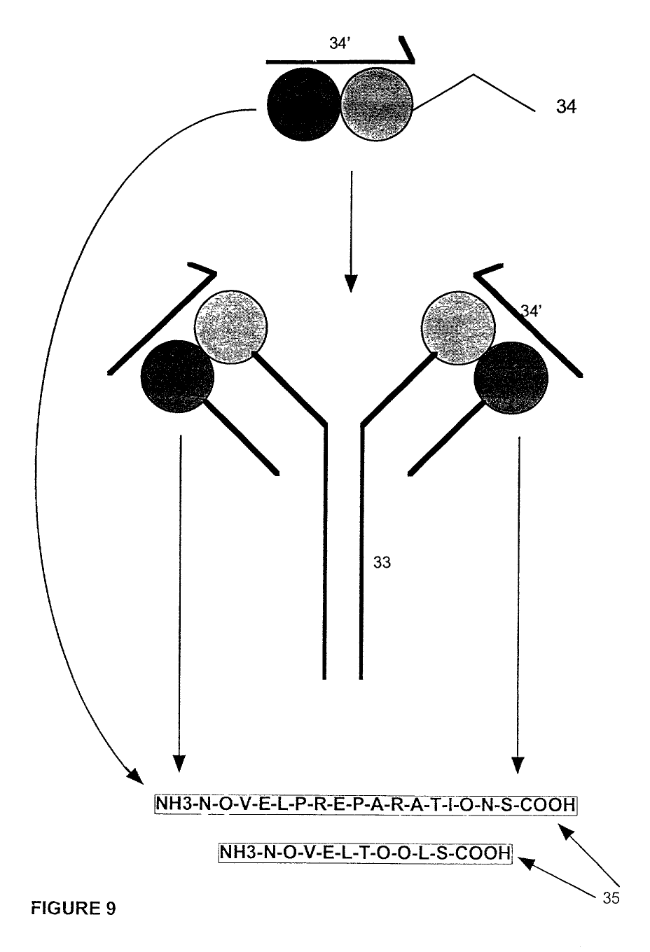

FIG. 9 schematically illustrates how the antibodies Ce3, R7 and R29 may be used to generate novel synthetic peptide therapeutics and/or tools;

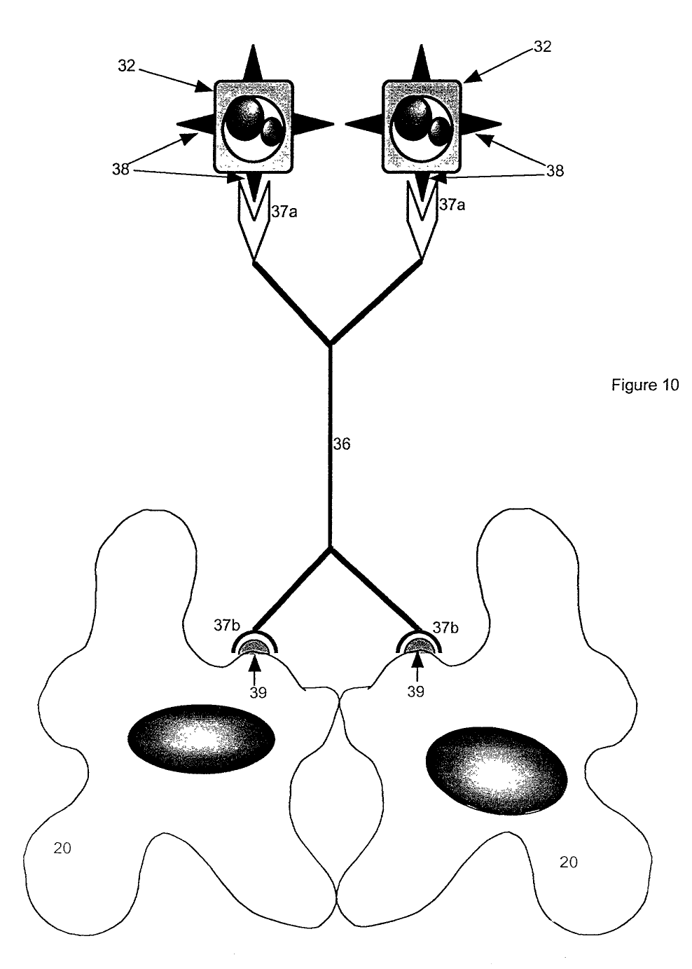

FIG. 10 schematically illustrates how the antibody Ce3 may be used as a bi-specific antibody;

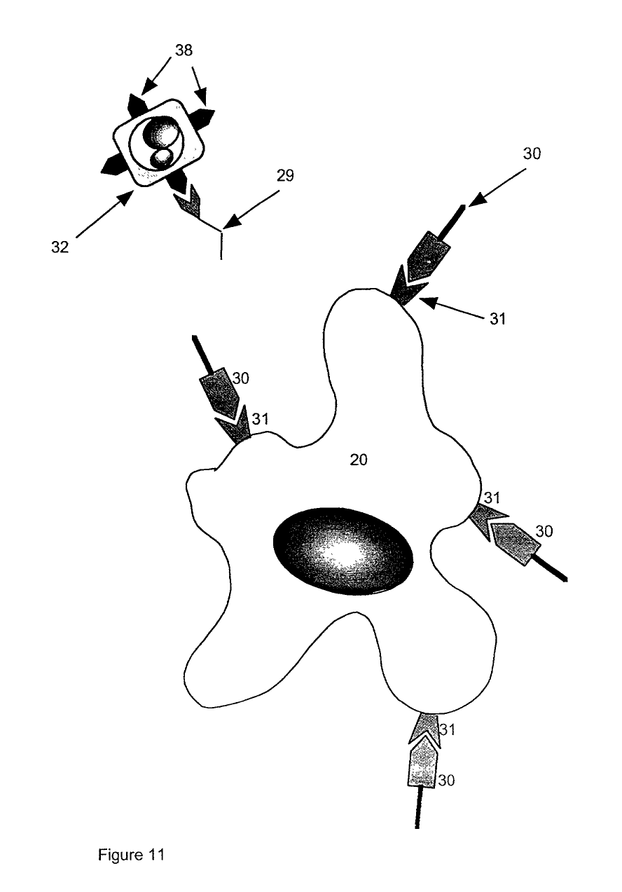

FIG. 11 schematically illustrates how the antibody Ce3 may be used to block the phagocytosis of ACs;

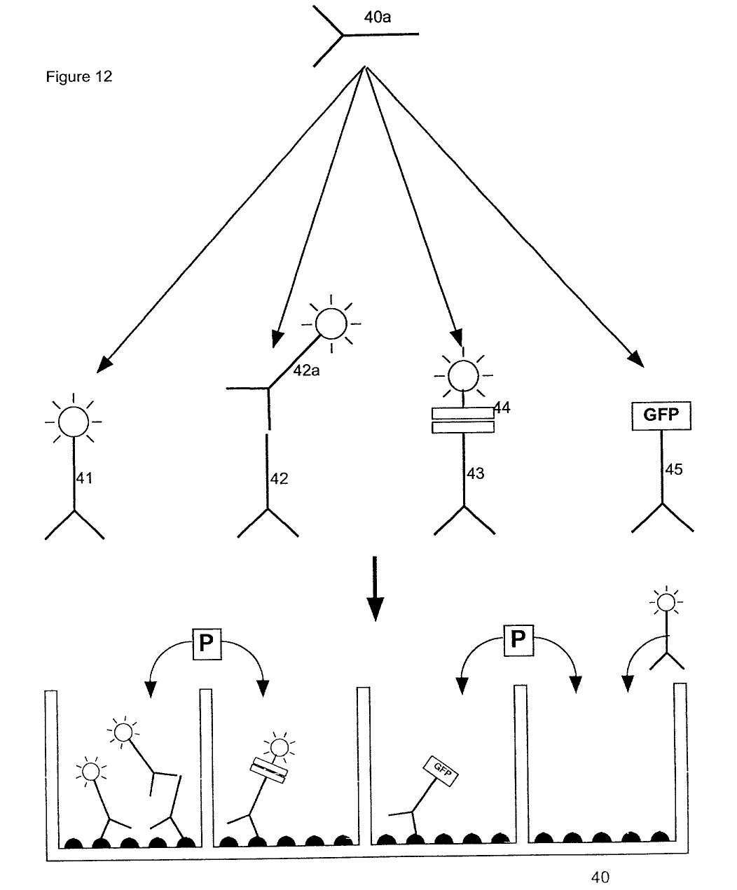

FIG. 12 schematically illustrates the use and manipulation of antibodies Ce3, R7 and R29 for High Throughput Screening (hereinafter termed HTS); and

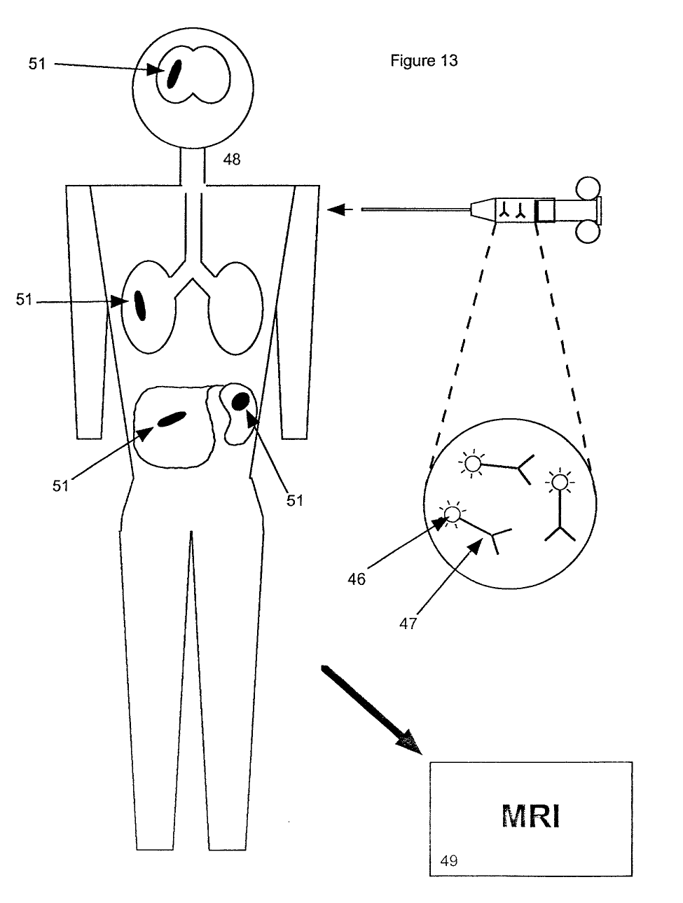

FIG. 13 schematically illustrates the use of the invention as a diagnostic/prognostic tool.

The initial stages of antibody or other reagent production using existing methodology is easily accomplished by persons with knowledge of the art. These stages for the selection and isolation of phage Ab reagents are outlined in FIG. 1a, FIG. 1b and FIG. 1c.

FIG. 1a schematically illustrates a semi-synthetic phage antibody display library Lib (Kruif et al., 1995), which comprises, in its most basic form, a phagemid 1 with the following elements: an antibiotic resistance gene for specific propagation in E. coli bacterial hosts 2; an E. coli replication origin 3; and an M13 filamentous phage element 4. The phagemid 1 also comprises for the expression of single chain variable fragments scFv, a leader sequence 5; many permutations of V_{H} and V_{L} genes and synthetic CDR3 regions to generate scFv diversity 6; an epitope tag (e.g. Myc tag) 7 for purification and detection of scFv fragments; an M13 phage coat protein Gene III 9; and an amber stop codon 8 situated after the scFv construct and before Gene III. In one specific example we may have 7 V_{L} genes and 49 V_{H} genes (in vitro), along with the synthetic CDR-3s generating the large diversity within the phage antibody library.

The phagemid 1 is transformed into a bacterial strain by a person of skill in the art using normal processes. There are two outcomes dependent on the strain of E. coli used (Hoogenboom et at., 1991). In transformed phagemids used in non-suppressor strains NS scFvs only are produced 10 as protein expression is terminated at the amber codon 8. Transformed phagemids used in suppressor strains S of E. coli with additional helper M13 phage HP produce phage expressing scFvs as a fusion to Gene III on the M13 phage 11, producing filamentous phage particles.

FIG. 1b schematically illustrates the selection of phage antibodies that bind to CD14, Negative selection is first carried out using cells that do not endogenously express CD14 12. In this case BJAB cells are used. Unbound phage antibodies 14 are then harvested and added, to BJAB cells that have previously been transfected and selected to stably express CD14 13 by methods known in the art. This positive selection involves binding phage 14 to cells expressing CD14 13. Phage 14a that are specific for CD14 15 are retained and non-specific phage 16 are washed away. The selection procedure is repeated to enrich the population of phage specific for CD14. Screening of isolated phage for clones of interest was carried out using immunofluorescence technology. Phage that stained BJAB expressing CD14 but not BJAB cells alone were identified as CD14 reactive phage. Such analysis raised a single phage of interest and this was named Ce3 (Cell-selected phage clone 3). Ce3 cDNA was deposited at the National Centre for Industrial, Food and Marine Bacteria, on Mar. 16, 2001, under accession number NCIMB 41088.

FIG. 1c schematically illustrates the selection of phage antibodies using ELISA technology. The well of a plate 17 is coated with a recombinant ICAM-3 Fc fusion protein 18 for negative selection of phage. Unbound phage 14 are harvested and added to a well 17a coated with a soluble, recombinant CD14-Fc fusion protein 19 for positive selection. Phage 14a that bind to CD14 19 are retained. This selection procedure is then repeated to enrich the population of phage that bind to CD14. Ø Abs that bound preferentially to CD14-Fc over ICAM 3 Fc or an IgG control are considered as candidates as CD14-reactive phage. This approach raised two phage of interest termed R7 and R29, (Recombinant-raised Ab7 or 29).

R7 and R29 cDNA was deposited at the National Centre for Industrial, Food and Marine Bacteria:, on Mar. 16, 2001, under Accession Nos. NCIMB 41086 and NCIMB 41087 respectively.

The preparation of phage antibodies from cDNA transformed into E. coli TG1 can be achieved as follows:

TG1 Cells containing DNA were grown overnight (12-16 hrs) with shaking in 2TY broth (10 g yeast extract, 16 g bacto-tryptone and 5 g sodium chloride [NaCl] per liter of water) supplemented with filter-sterilised glucose (5% w/v) and ampicillin (100 μg/ml). The following day 10 μl of the overnight cultures were added to 100 μl of fresh 2TY with 100 μl/ml of ampicillin and 5% (w/v) of glucose in a 96 well plate and left for 2 hours at 37° C. with shaking. 10 μl of 2TY was then added containing 1×10⁹ plaque forming units (pfu) of VCS-M13 helper-phage (Pharmacia-Biotech).

The 96 well plate was then incubated for 1 hour at 37° C. without shaking. The 96 well plate was then centrifuged for 20 minutes at 3000 rpm (to pellet the bacteria) and supernatants were discarded. Pellets in the wells of the 96 well plate were resuspended in 250 μl of 2TY with 100 μg/ml of ampicillin and 25 μg/ml of kanamycin (no glucose).

The plate was then further incubated at 30° C., overnight, with shaking.

The following day the 96 well plate was centrifuged for 20 minutes at 3000 rpm and the supernatants containing the phage antibody were stored at 4° C.

If larger quantities of the phage antibodies are required, the phage can be purified using polyethylene glycol (PEG)/NaCl precipitations from larger culture volumes as follows:

An overnight culture (250 ml) was centrifuged at 8000 rpm for 10 minutes. ⅕ of the volume 20% (w/v) PEG 6000 in NaCl (2.5M) was added to the supernatent, mixed and incubated for 1 hour on ice. The culture was then centrifuged at 8000 rpm for 30 minutes, and the supernatent discarded. The pellet was re-suspended in 40 ml of phosphate buffered saline (PBS) and 8 ml of PEG 6000/NaCl and incubated for 30 mins on ice. The cultures were then further centrifuged at 8000 rpm for 30 minutes. The supernatent discarded and the pellet re-suspended in 190 mls of 1% (w/v) bovine serum albumin (BSA) in PBS and filtered through a 0.45 μm filter to remove bacteria was stored at 4° C.

Soluble single chain FV fragments can be produced by the transformation of a plasmid containing the cDNA isolated from TG1 cells as follows:

TG1 cells containing the cDNA for the single chain FV fragments that had been cultured overnight at 37° C. with shaking in a conical flask containing 2TY with 5% (w/v) glucose and 100 μg/ml of ampicillin were subjected to a “mini-prep” procedure to isolate plasmid DNA (by methods known in the art). The “mini-prep'd” DNA was subsequently transformed into competent SVF110 cells (using methods known in the art) and plated onto TYE (5 g yeast extract, 10 g bacto-tryptone, 8 g NaCl and 15 g bacto-agar per liter of water) plates with 100 μg/ml of ampicillin, 5% (w/v) glucose and incubated at 37° C. overnight. Colonies on the plate were scraped into 2TY with 100 μg/ml of ampicillin, 10 μg/ml of tetracycline and 0.1% glucose, and incubated at 25° C. with shaking until an optical density of 0.5 at 600 nm had been reached. IPTG (isopropyl-β,D-thiogalactoside) was then added, to a final concentration of 1 mM, to the culture. The culture was incubated at 25° C. overnight. The following day the culture was centrifuged for 20 minutes at 3000 rpm at 4° C., and the supernatent discarded. The pellet was re-suspended in 1% of the original volume in cold TES (0.2M Tris -HCl, 0.5 mM ethylenediaminetetraacetic acid [EDTA] and 0.5M sucrose). 1.5% of the original volume of ice-cold TES was then added. The re-suspended culture was incubated on ice for 30 minutes, and then centrifuged at 8000 rpm for 20 minutes at 4° C. The scFv-containing supernatants were transferred to a clean tube and stored at −20° C.

FIG. 2 shows the results of three tests: an ELISA where CD14-Fc, ICAM3 Fc and IgG were coated onto respective 96 well plates. 63D3 is a CD14 specific mAb. 63D3 binds to soluble recombinant CD14 as shown on the graph by an increase in optical density at 492 nm and not to ICAM-3 or IgG, however Ce3 binds to no significant degree to soluble, recombinant CD14 yet R7 and R29 do, as shown by an increased optical density (OD) reading at 492 nm, compared with the OD of Ce3 binding to CD14 at 492 nm. R7 and R29 are not binding to the Fe region of the fusion protein as indicated by the lack of reactivity to IgG confirmed by the low OD reading at 492 nm. 63D3 has not been shown to block AC clearance or block the pro-inflammatory response and so may bind to a different epitope on CD14.

FIG. 3 shows FLOW CYTOMETRIC profile results of a range of Abs on two cell types: BJAB (on the left off the page) and BJAB/CD14, on the right of the page; (where CD14 is exogenously expressed by the BJAB/CD14 cells on the cell surface) 63D3 is an anti-CD14 Ab tat shows good staining to CD14-expressing cells B(i) as indicated by a peak shift PS relative to a control peak C. Similar results are seen for Ce3 B(ii) indicating that this phage Ab as a scFv (though also as a whole phage Ab) binds to cell-associated CD14 as demonstrated by a peak shift PS. Similar results are seen using whole phage Abs. R7 and R29 show no binding to cell-associated CD14 (Biii and Biv respectively).

FIG. 4 shows similar results with two other cell types (CHO on the right and K562 on the left) that are exogenously expressing CD14. 63D3 is an anti-CD14 Ab that shows good staining of CD14-expressing cells as indicated by a peak shift PS relative to a control peak C. Similar results are seen for Ce3 indicating that this Ab as scFv (though also as a whole phage Ab) binds to cell-associated CD14 as demonstrated by a peak shift PS relative to a control peak (Aii and Bii). R7 and R29 show no binding to cell-associated CD14 (Aiii, iv and Biii, iv).

FIG. 5 shows staining of a cell line (THP-1) stimulated to express CD14 with dihydroxyvitamin D₃ whilst FIG. 6 shows similar results with primary monocytes, granulocytes and lymphocytes i.e. strong staining of monocytes, weaker staining of granulocytes indicated by a peak shift PS relative to a control peak C. and no straining of lymphocytes, indicated by no peak shift, from fresh blood. This staining pattern is consistent with the known staining patterns of CD14 on these cell types.

In summary, any cell type that expresses CD14 (either endogenously or exogenously) appears to be stained by the reagent Ce3: we did not find one that was not.

Ce3 also has interesting biological functions. That is to say it is capable of blocking the clearance of apoptotic cells by macrophages (FIG. 7).

FIG. 7A shows the results of the inhibition of interaction (i.e. binding and phagocytosis) of apoptotic B-cells with macrophages. The addition of 63D3 does not inhibit the percentage of macrophages interacting with the apoptotic B-cells compared to the control of macrophages alone. The addition of 61D3 does inhibit the percentage of macrophages interacting with apoptotic B cells compared to the control macrophages alone in line with observations by Devitt et al. (1998). When Ce3 is added there is a decrease in the percentage of macrophages able to interact with apoptotic B-cells indicated by a decrease in the eight of the bar on the graph.

FIG. 7B shows the results of the effects of Ce3 on the interaction of macrophages with apoptotic B cells. Neat Ce3 inhibits the percentage of macrophages that can interact with apoptotic B-cells as indicated by a decrease in the height of the bar on the graph. A 1 in 4 dilution of Ce3 results in no change in the percentage of macrophages that can interact with apoptotic B-cells.

FIG. 8 schematically illustrates Ce3 30 in accordance with the invention binding to CD14 31 on macrophages 20. It is known that ACs binding to macrophages effectively reduces (if not switches off) the inflammatory response mediated by CD14 elicited by macrophages. Ce3 30 can be (mimicing AC ligands) employed as a specific anti-inflammatory preparation 30′ which when administered to mammals (e.g. humans) can alleviate inflammation (I). One possible use is as a treatment for rheumatoid arthritis (as inflammation in joints causes severe pain).

Ce3 could also be conjugated with a cytotoxic moiety and targeted to unwanted CD14-expressing cells as might occur in some malignancies of myeloid lineage cells. As the species specificity can be altered to suit the subject/patient, toxicity is greatly reduced.

Ce3 may also be used to block lipopolysaccharide (LPS)-induced inflammation by Gram-negative bacteria. This inhibition of inflammation may be hypothesised to be achieved in either or both of two) ways:

(i) Ce3 may mimic an AC ligand and cause the anti-inflammatory effects associated with AC clearance i.e. by production of Transforming Growth Factor-⊕1 (TGF-β1); or

(ii) Ce3 may work in a like way to the antibody 61D3 (FIG. 7) that is to say that Ce3 and 61D3 may behave in an identical fashion in all the assays carried out, i.e, they both bind to CD14, and they both inhibit AC clearance by macrophages. It may be that Ce3 acts in a similar manner to 61D3 as described previously (Devitt et al, 1998) in which it was suggested that 61D3 blocked inflammation by blocking the binding of LPS to CD14.

It will be appreciated that the above hypothesis as to the mechanism of possible working are (1) non-limiting to the invention, and (2) constructed after the fact that Ce3 does have the interesting requisite properties that had been established. Such hypotheses were not possible until it was known to work.

FIG. 9 shows the case of peptide preparations as therapeutics and tools to study receptor—ligand interactions. It is known that antibodies can act as ligand mimetics. We reverse engineer the Ø-Ab Ce3 (phage or scFv) 34 into a whole antibody 33. It is then possible to sequence the DNA coding for the scFv to predict the amino acid sequence of the binding site (variable regions) of these antibodies 34′. Obtaining the sequence of the Ce3 will yield the amino acid sequence of the ligand mimetic element of Ce3 34.

This knowledge can be used to generate novel synthetic peptide preparations 35.

Using the synthetic peptide 35 it is possible to generate novel ligand-receptor interactions such, as to perturb physiological receptor-ligand interactions using the synthetic peptides.

It will be appreciated that the preparations can be used in humans and/or in alternative species to humans. Preferably the preparation can be used in human cell lines/subjects/assays.

As illustrated in FIG. 10, Ce3 can be used to produce a bi-specific antibody. The bi-specific antibody 36 is shown comprising different idiotypes 37a and 37b. The idiotypes recognise different ligands 38 present on ACs 32 and CD14 receptors 39 present on macrophages 20. The bi-specific antibodies 36 will then act an inducer of clearance of said ACs 32 by tending to cause MØ to be present near to ACs. The application of bi-specific antibodies 36 has utility in assays or therapeutics that require an increase in the clearing of unwanted cells generally e.g. neoplasias. Alternatively bi-specific antibodies could be used to provide the removal of bacteria that would normally bind to CD14 and elicit an inflammatory immune response. The bi-specific antibody would bind phagocytes at one end (via CD14) and bacteria at the other end (e.g. a bacterium-specific Ab that could be tailored to individual species of genera e.g. the major outer membrane protein of Chlamydia or more generally LPS, peptidoglycan or any surface-exposed moiety).

It will be appreciated that bi-specific antibodies can be utilised for other aspects of apoptosis research.

It will be appreciated that bi-specific antibodies can be utilised to facilitate the removal of unwanted cells, proteins or other structures if the ‘other’ end of the Ab can react with said unwanted structure.

Moreover, bi-specific antibodies can associate a label or therapeutic substance with phagocyte if the “other” end of the bi-specific antibody binds to a label or therapeutic substance.

Once again, bi-specific antibodies can be used in alternative species, but a primary one is in human cell lines/subjects. It will also be appreciated that we do not wish to restrict ourselves to bi-specific multi-specific could be used. Multi-specific could be advantageous in situations where two or more substances need to be in the vicinity of phagocyte to influence its apoptotic cell clearance activity (or other activity as suggested above).

Blocking the clearance of ACs is desirable in many disorders e.g. neurodegenerative disorders and derivatives thereof, strokes, and in acquired immunodeficiency syndrome (AIDS) or other condition where cells are deleted and removed inappropriately. Illustrated in FIG. 11 is the use of Ce3 made in accordance with the invention to block clearance of ACs 32 by macrophage cells 20. This coupled with agents to block other events in the apoptosis programme could result in otherwise deleted cells remaining live and functional in situ.

Applications of antibody Ce3 in AIDS and neurodegenerative disorders comprising; for example, Creutzfeld-Jacob disease, Huntingtons disease, Alzheimer's disease etc, possibly in conjunction with other therapeutic agents or derivative thereof, may lead to a decrease in clearance of said ACs and to a slowing in progression of said disorders. The invention may be applied to a number of different species (after modification of the antibodies by persons skilled in the art) different cell lines, and to a number of species in vivo, including human subjects/cell lines.

Illustrated in FIG. 12 is a screening method employing the invention to screen for CD14. Such an assay may be used for a variety of situations from the low throughput research applications to and including (though not limited to) high-throughput screening assays. As such the Abs in accordance with this invention may be used on any platform where detection of the Ab or reagent is made possible.

This mode of employment of said invention will provide a non-subjective assessment of cells expressing CD14 and replace subjective recording by human eye, or other low throughput assays. HTS may be used to identify new pharmaceutical preparations responsible for modulating CD14 expression by cells. This screen for novel pharmaceutical preparations will involve the use of the antibody Ce3 after prior application of pharmaceutical preparations to cells present on a carrier 40 illustrated by FIG. 12. The carrier may take the form of a gel or a well in a plate or other suitable format for assay.

Marriage of Ce3 40a to screening techniques will provide a fast, efficient and specific assay format for studying CD14 and identity of novel pharmaceutical preparations P.

For accurate data acquisition from screening tests Ce3 may need to be manipulated so as such to comprise a preferably water-soluble dye e.g. fluorescein isothiocyanate or derivatives thereof 41, or other marker.

This is, of course, to make them detectable by the observer and as such any detectable marker may be used with Abs and other reagents from this invention.

Alternatively Ce3 may be used as a primary detection agent 42 and detection achieved with a labelled secondary detection reagent 42a (the secondary detection reagent binds to the primary agent) as appropriate. The Ab or other reagent 42 may contain a tag e.g. an epitope tag such as a myc tag to which the secondary reagent binds.

Ce3 maybe biotinylated 43 for detection by a streptavidin (or equivalent) conjugate of a label such as peroxidase or derivatives thereof 44. Preferably said streptavidin-conjugate 44 could comprise a fluorescent dye such as aforementioned water-soluble dyes or derivatives thereof or other detectable label.

Ce3 may also be engineered to comprise an endogenous fluorescent tag 45 eg. Green Fluorescent Protein or derivatives thereof or other detectable tag.

Thus said invention when married with screening methods will provide efficient and specific ways for identification of pharmaceutical preparations of possible therapeutic utility.

It will be appreciated that said application of screening tests with respect to CD14 research will be applied to various elements of CD14 research. For example one may want to measure kinetics of CD14 expression in a desired cell line using many known/unknown preparations P. The kinetics of specific AC ligand exposure during apoptosis could be assessed on exposure to the preparations P. Such assays need not be restricted to HTS and may also be applied to any format where the Ab or other reagents may be detectable.

It will also be appreciated that Ce3 can be modified to charge the species type of the original phage antibody i.e. to change it from an anti-CD14 phage Ab or an anti-CD14-scFv to an anti-CD14 whole human (or other species) antibody whilst retaining the specificity of the original phage Ab or scFv antibody. This allows the scFv antibody after prior modification to be detected by a wide variety of species-specific secondary reagents. This reverse engineering could be performed to transform scFv's into various different species whole antibodies. Thus providing more assay diversity in screening processes. Additionally such humanised reagents (or other species optimised reagents), for example, will be of more use in vivo, as they will be less likely to elicit undesirable immune responses in the recipient.

The present invention can be used to produce a novel diagnostic and prognostic marker, as discussed above.

Said invention may be used with any appropriate, detectable reagent and matching detection technology.

Ce3 is capable of blocking AC-clearance by phagocytes in addition to detecting phagocytes. Although inhibiting apoptotic-cell clearance does not at first sight seem helpful in cancer treatment, it may be useful to encourage the development of immunity towards those cells, thus promoting anti-tumour immunity. Our reagents will be capable of more than blocking cell clearance. Additionally, to the diagnostic/prognostic use they can promote clearance (bi-specific approach for example) and promote anti-tumour capabilities (e.g. by using an inflammatory tag on our reagents so as to promote natural immune responses at the site of tumours where phagocytes accumulate and engulfment by Mø).

Non-invasive monitoring of patients who have received chemotherapeutic agents is a technology that is desirable. A non-invasive technique would alleviate strain on health services world wide. A relatively non-invasive procedure may be undertaken by appropriately trained nursing staff. Chemotherapeutic agents often target rapidly dividing cells to cause massive cell death. This killing of rapidly dividing cells accounts for many common side affects of chemotherapy e.g. alopecia, and a compromised immune system. Patients may be subjected to several different protocols before the correct protocol of agents is found. A wait of some time is necessary before beginning treatment with a first agent and performing a test e.g. a biopsy to see if it is working. Current screening procedures e.g. magnetic resonance imagine (MRI) and positron emission topography (PET) do not provide a non-invasive assessment as to the efficacy of said chemotherapeutic agents. These techniques may be non-invasive, but they do not gives an accurate indication of the efficiency of a drug effect during a course of chemotherapy and therefore for this reason MRI and PET do not traditionally provide a non-invasive assessment as biopsies are still the mainstay of monitoring a chemotherapy course.

As illustrated in FIG. 13, Ce3 47 can be manipulated to include an element 46 detectable to MRI technology 49. Neoplasias may have a high level of apoptosis occurring within them (with more aggressive tumours often having then highest levels of apoptosis). This is associated with infiltration of phagocytes expressing CD14. Ce3 may be administered to a patient 48 with neoplasia 51 to monitor, via MRI 49, the efficacy of administered chemotherapeutic agents in a non-invasive manner. The concentration of MRI tagged, Ce3 at the desired site of study 51 could be ascertained within days (instead of weeks), allowing the selected therapeutic treatment to be changed much faster if it did not work. Induction of death at a site would be accompanied by increased phagocyte infiltration (and therefore increased CD14-expressing cells).

As Ce3 may be a recombinant human antibody, toxicity is reduced. Preferably said invention could be manipulated so as to comprise an anti-neoplastic preparation (the preparation comprising a drug or derivative thereof) directed against specific neoplasias utilising the cell lineage specificity of said invention. This may eliminate or reduce widespread side effects of said preparation.

Preferably said invention could also be employed to non-invasively monitor other disorders involving phagocyte accumulation. Preferably said invention could monitor said disorders anywhere in the body, Monitored disorders may comprise, for example, Tuberculosis, Sarcoidosis, Crohn's Disease, Rheumatoid Arthritis, Cancer, and any other disease in which monitoring the levels of CD14 expressing cells may be useful.

Preferably screening will be carried out on human subject but it will be appreciated that screening can also be carried out in other species.

The present invention can be used to produce a novel diagnostic and prognostic marker, as discussed above,

Said invention may be used with any appropriate, detectable reagent and matching detection technology.

Products of said invention may be capable of blocking AC-clearance by phagocytes in addition to detecting phagocytes. Although inhibiting apoptotic-cell clearance does not at first sight seem helpful in cancer treatment, it may be useful to encourage the development of immunity towards those cells, thus promoting anti-tumour immunity. Our reagents will be capable of more than blocking cell clearance. Additionally, to the diagnostic/prognostic use they can promote clearance (bi-specific approach for example) and promote anti-tumour capabilities (e.g. by using an inflammatory tag on our reagents so as to promote natural immune responses at the site of tumours and engulfment by Mø).

As R7 and R29 only bind to soluble, recombinant CD14 the se Ø-Abs may be used in detection methods in research labs e.g. detecting soluble CD14 on western blots, dot blots, slot blots and ELISA and fluid samples.

R7 and R29 may be labelled with an enzyme e.g. peroxidase or another label to aid detection. Alternatively they may be detected using a secondary antibody labelled with peroxidase or another label. R7 and R29 may be detected with labelled secondary and tertiary or more antibodies, or combinations of labelled and unlabelled antibodies.

1. A method of detecting CD14, comprising using at least one of Ce3 or R7 or R29.

2. A method of detecting CD14 comprising recognising CD14 using at least one antibody capable of being expressed from the cDNA isolated from the bacterial strains having Accession Nos. NCIMB 41086, NCIMB 41087 or NCIMB 41088.

3. A method according to claim 1 in which the Ce3 or R7 or R29 is a monoclonal antibody.

4. A method according to claim 2 in which the antibodies is Ce3 which can be expressed from the cDNA isolated from the bacterial strain having the Accession No. NCIMB 41088.

5. A method according to claim 2 in which the antibody is R7 which can be expressed from the cDNA isolated from the bacterial strain having the Accession No. NCIMB 41086.

6. A method according to claim 2 in which the antibody is R29 which can be expressed from the cDNA isolated from the bacterial strain having the Accession No. NCIMB 41087.

7. A method according to claim 4 comprising any antibody with 70-100% sequence identity and/or homology with Ce3.

8. A method according to claim 7 comprising any antibody with 90-100% sequence identity and/or homology with Ce3.

9. A method according to claim 5 comprising any antibody with 70-100% sequence identity and/or homology with R7.

10. A method according to claim 9 comprising any antibody with 90-100% sequence identity and/or homology with R77.

11. A method according to claim 6 comprising any antibody with 70-100% sequence identity and/or homology with R29.

12. A method according to claim 11 comprising any antibody with 90-100% sequence identity and/or homology with R29.

13. A method according to claim 2 in which the antibodies are produced or derived from a phage antibody library.

14. A method according to claim 7 in which the antibodies are a single chain FV molecule derived from phage Ab.

15. A method according to claim 7 in which a phage Ab or single chain phage antibody is reverse engineered to a conventional whole antibody of a specified isotype.

16. A method according to claim 2 in which a single chain phage antibody is reversed engineered to a conventional whole antibody of a specified isotype.

17. A method according to claim 2 in which the phage antibody Ce3 is specific for CD14 expressed on the surface of cells in vitro and in vivo.

18. A method according to claim 2 in which the phage antibody R7 or R29 is specific for soluble CD14.

19. A method according to claim 13 which comprises using a semi-synthetic phage antibody display library which comprises a phagemid with one or more of the following elements:
(i) an E. coli replication origin; and
(ii) an M13 filamentous phage elements.

20. A method according to claim 19 in which the phagemid also comprises, for the expression of single chain variable fragments, one or more of:
(i) a leader sequence;
(ii) a large number of permutations of V_{H} and/or V_{I} genes and/or synthetic CDR3 regions;
(iii) a Myc tag for purification and/or detection of scFv fragments;
(iv) an M13 phage coat protein Gene III;
(v) an amber or ochre or opal stop codon situated after the scFv construct and before Gene III.

21. A method according to claim 7 in which the phage library is transformed into a suitable suppressor strain of bacteria to produce filamentous phage expressing scFv's, preferably as a fusion to Gene III.

22. A method of testing an animal or sample from an animal comprising applying Ce3 to cells of interest or a test area and observing a signal from the test area, said Ce3 being directed to CD14 on phagocytes.

23. A method according to claim 2 comprising applying Ce3 to a histological tissue sample.

24. A method according to claim 22 performed in vivo, and also comprising imaging a portion of the animal's body, a detectable signal being provided by the marker.

25. A diagnostic or screening test reagent for use in testing an animal or sample from an animal, the reagent comprising Ce3, said Ce3 being directed to and reactive with CD14 on phagocytes CD14 being involved in and having a marker or a site to bind a marker to provide a signal to indicate the presence of Ce3.

26. The use of Ce3 or R7 or R29 in the preparation of a diagnostic reagent, said Ce3 being directed towards and reactive with CD14 on phagocytes and R7 and R29 are reactive with soluble CD14, and Ce3 or R7 or R29 having a marker or a site to bind a marker, to provide a signal to indicate the presence of Ce3 or R29.

27. The use of antibody Ce3 or R7 or R29 to produce an imaging reagent, in which Ce3 is directed against and reactive with CD14 on phagocytes and R7 and R29 are reactive with soluble CD14, the antibody having a marker or a site to bind a marker, to provide a signal to indicate the presence of Ce3 or R7 or R29.

28. The use of Ce3 in the preparation of a medicament for the treatment of a disease or disorder.

29. The use according to claim 28 in which isolated or purified Ce3 is used in the manufacture of a medicament for the treatment of a disease or disorder, Ce3 being directed to a reactive with CD14 on phagocytes.

30. The use according to claim 29 in which Ce3 is capable of being expressed from the cDNA isolated from the bacterial strain having Accession No. 41088.

31. A method of treating an inflammatory disease, condition or disorder comprising using Ce3 to treat disease, condition or disorder.

32. The use of a reagent according to claim 26, which reagent comprises an indicator for a disease or disorder from the list:
cancer;
autoimmune disease (e.g. systemic lupus erythematosus);
stroke;
infertility;
myocardial infarction;
tuberculosis;
sarcoidosis;
Crohn's disease;
rheumatoid arthritis;
any disease where macrophage (or other CD14-expressing cell) persistence or accumulation presence is implicated in the aetiology of the disease;
any condition where suppression of inflammation may be an effective therapy;
any condition where modulation of function of CD14-expressing cells may be an effective therapy.

33. A method of prognosis for a patient receiving a treatment for a disease or disorder, the method comprising administering a treatment for the disease or disorder; waiting a time sufficient for the treatment to have a measurable effect; administering an imageable or detectable reagent, and in which the imageable or detectable reagent comprises Ce3; Ce3 having a detectable signal or have a binding site for a signal-generating marker that is also administered, imaging or otherwise analysing the signal from the site of the patient that has the disorder or disease to evaluate the level of apoptosis at the site, thereby enabling a prognosis for the patient to be evaluated by evaluating the efficacy of the treatment.

34. A method according to claim 33 in which the imaged or otherwise analysed results from before treatment and after a treatment compared in order to assist in determining whether the treatment is effective.

35. A method according to claim 33 in which instead of practising the prognosis indication on the body of a patient it is practised in vitro on a tissue sample, blood, or other fluid, sample, or biological or clinical sample.

36. The use of at least one of the antibodies Ce3, R7 or R29, or other substances derived from them in the identification of non-inflammatory drugs.

37. The use of Ce3, R7 or R29 or derived substances, to attach phagocytes to apoptotic cells or other cells, the antibodies, comprising bispecific antibodies and the derived substances comprising bispecific derived substances.

38. A method of detecting phagocytes comprising ensuring that the phagocytes are exposed to a reagent which contains at least one of the antibodies Ce3, R7, R29; and a marker associated with the antibody.

39. A method according to claim 38 in which the observed signal is imaged or otherwise detected, thereby imaging/detecting the phagocytes, or region of the phagocytes which has the signal, or area where the phagocytes are located.

40. A method of turning off the inflammatory response in phagocytes comprising having the phagocytes encounter Ce3 which is directed to and reactive with CD14.

41. A method according to claim 38 comprising detecting phagocyte accumulation.

42. A method in which no site-specific antibodies are used, instead non-antibody substances are used.

43. A method according to claim 2 in which R7 or R29 is used to detect soluble CD14.

44. A method according to claim 2 in which R7 and R29 is used to detect soluble CD14 and R7 or R29 is (i) labelled with peroxidase or another label to aid detection, or (ii) is detected using a secondary antibody labelled with a label.

45. A CD14-reactive reagent comprising at least one of Ce3; or R7; or R29.

46. A reagent according to claim 45 which has substantially no additional antibodies which have no CD14 reactivity.

47. A reagent according to claim 46 which is substantially free of other biologically active materials which influence the activity of CD14 with Ce3, R7 or R29.

48. A reagent according to claim 45 which comprises one of (i) a diagnostic reagent; (ii) a therapeutic agent.

49. Isolated Ce3, R7, or R29.

50. A vector, or plasmid, or phagemid, having the DNA/cDNA of NCIMB 41088, or NCIMB 41086 or NCIMB 41087, and substantially no other antibody nucleotide sequences an expression position.
